(19) United States
(12) Patent Application Publication
(10) Pub. No.: US 2010/0043672 A1
(43) Pub. Date: Feb. 25, 2010

(54) METHOD OF FINISHING ORGANIC PIGMENTS

(75) Inventors: Joachim Jesse, Weisenheim (DE); Benno Sens, Neustadt (DE); Andres Carlos Garcia Espino, Mannheim (DE); Richard Van Gelder, Speyer (DE); Matthias Klueglein, Ludwigshafen (DE); Ines Pietsch, Speyer (DE); Wolfgang Best, Freinsheim (DE); Rainer Henning, Freinsheim (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: Basf Se, Ludwigshafen (DE)

(21) Appl. No.: 12/595,549

(22) PCT Filed: Apr. 11, 2008
(86) PCT No.: PCT/EP2008/054425

§ 371 Date: Oct. 12, 2009

(30) Foreign Application Priority Data
Apr. 13, 2007 (EP) 07106104.8

(51) Int. Cl. C08K 5/23; C07C 245/04

(52) U.S. Cl. 106/493; 534/563

(57) ABSTRACT

The invention relates to a method of finishing an organic pigment that involves dissolving or dispersing the pigment in an aqueous solvent or a mineral acid and crystallizing the pigment from the solution or dispersion in the presence of a crystallization modifier, and subsequently isolating the pigment as a solid, wherein the crystallization modifier is a condensation product containing sulfonate groups and formed from at least one hydroxyarylsulfonic acid and/or at least one hydroxydiaryl sulfone compound and at least one aliphatic aldehyde having 1-6 C atoms, optionally urea, and an alkali metal sulfite if appropriate, or is a mixture of such condensation products.

The invention relates to a method of finishing organic pigments using condensation products that contain sulfonate groups and are based on hydroxyarylsulfonic acids or hydroxydiaryl sulfone compounds as crystallization modifiers, to the resultant organic pigments themselves, and to the use of the condensation products as crystallization modifiers for finishing organic pigments. Sulfonate groups below refer both to the (acidic) sulfonic acid group —SO₃H and to its salt —SO₃M (M=metal; ammonium).

At the synthesis stage, organic pigments are frequently obtained in a coarsely crystalline form with a very heterogeneous particle size distribution. To convert them into a coloristically valuable pigment form suitable for application, therefore, the crude pigments are typically subjected to a finishing operation.

Known for these purposes is the grinding of the crude pigment and subsequent recrystallization of the millbase from an organic solvent, or else the grinding is carried out as a wet grinding operation in aqueous suspension in ballmills with high-speed stirrer mechanisms. The processes described do achieve particle size reduction, in some cases involving lengthy effort, and hence an improvement in the coloristic properties of the pigments; the particle size of the pigments obtained, however, is difficult to control, and the pigments frequently have a particle size distribution which is too broad and hence inadequate for a range of applications, for example, as colorants for paints and in inkjet inks.

WO 02/00643 discloses a method of finishing crude quinophthalone pigments that involves subjecting the as-synthesized crude pigment to grinding in the absence of grinding assistants and then crystallizing the resulting millbase in the presence of a quinophthalone derivative in an organic solvent or in a mixture of organic solvent and water. Derivatives specified include, for example, sulfonic acid derivatives of the quinophthalone pigments.

WO 2004/048482 discloses a method of finishing organic pigments that involves dissolving the pigment in concentrated sulfuric acid and mixing the sulfuric acid solution with water in the presence of a condensation product of naphthalenesulfonic acid and formaldehyde as crystallization modifier. The crystallization modifier in that case is added to the sulfuric acid pigment solution or is generated in that solution in situ by reacting 1- and 2-naphthalenesulfonic acid with formaldehyde.

It is an object of the invention to provide further advantageous methods of finishing organic pigments that yield pigments having very good coloristic properties.

This object is achieved by means of a method of finishing an organic pigment that involves dissolving or dispersing the pigment in an aqueous solvent or a mineral acid and crystallizing the pigment from the solution or dispersion in the presence of a crystallization modifier, and subsequently isolating the pigment as a solid, wherein the crystallization modifier is a condensation product containing sulfonate groups and formed from at least one hydroxyarylsulfonic acid and/or at least one hydroxydiaryl sulfone compound and at least one aliphatic aldehyde having 1-6 C atoms, optionally urea, and an alkali metal sulfite if appropriate, or is a mixture of such condensation products.

Crystallizing in the sense of the present invention is not only the precipitating of the pigment as a solid from its solution, but also the finishing of the pigment, which goes hand in hand with partial recrystallization of the pigment.

The crystallization modifier may be a condensation product of one or more different hydroxyarylsulfonic acids and one or more different aliphatic aldehydes with 1 to 6 C atoms. Generally speaking, condensation is carried out with 0.25 to 4 mol of aliphatic aldehyde per mole of hydroxyaryl units present. If urea is used as well, it is generally employed in amounts of 0.25 to 4 mol of urea per mole of hydroxyaryl units present. Besides hydroxyarylsulfonic acid it is possible for one or more different hydroxydiaryl sulfone compounds to be present in cocondensed form in the condensation product. The preparation of the condensation product can additionally be carried out in the presence of an alkali metal sulfite, preferably sodium sulfite, in which case further sulfonate groups—in addition to the sulfonate groups introduced by the hydroxyarylsulfonic acid—are introduced into the condensation product.

The crystallization modifier may be a condensation product of one or more different hydroxydiaryl sulfone compounds, one or more different aliphatic aldehydes with 1 to 6 C atoms, and an alkali metal sulfite, preferably sodium sulfite. In this case the sulfonate groups are introduced as alkylsulfonate groups into the condensation product. Generally speaking, the hydroxydiaryl sulfone compound is reacted with 0.5 to 5 mol of the aliphatic aldehyde and 0.4 to 2 mol of the alkali metal sulfite per mole of hydroxydiaryl sulfone compound.

Examples of suitable hydroxyarylsulfonic acids which can be used to prepare the crystallization modifiers used in accordance with the invention are hydroxyphenylsulfonic acids or hydroxynaphthylsulfonic acids. These acids may also contain two or more hydroxyl groups. A preferred hydroxyarylsulfonic acid is phenolsulfonic acid.

Examples of suitable hydroxydiaryl sulfone compounds which can be used to prepare the condensation products employed in accordance with the invention are dihydroxydiphenyl sulfones or, more generally, polyhydroxydiphenyl sulfones, and also dihydroxydinaphthyl sulfones or, more generally, polyhydroxydinaphthyl sulfones. A preferred hydroxydiaryl sulfone compound is dihydroxydiphenyl sulfone (DHDPS).

Generally speaking, hydroxyarylsulfonic acids and hydroxydiaryl sulfones are prepared by sulfonating the corresponding hydroxyaryl compounds, such as naphthols, phenol or polyphenols, with concentrated sulfuric acid or oleum. This generally produces mixtures of hydroxyarylsulfonic acids and hydroxydiaryl sulfones.

One preferred embodiment of the method of the invention uses a crystallization modifier obtainable by reacting phenol with concentrated sulfuric acid, with oleum with an SO₃ content of 20% to 65% by weight or with a mixture of sulfuric acid and oleum, the molar ratio of total sulfuric acid, calculated as SO₃, to phenol being 0.7:1 to 1.5:1, at a temperature of 100 to 180° C., to give a mixture comprising phenolsulfonic acid, dihydroxydiphenyl sulfone, and sulfuric acid, or prepares a corresponding mixture by blending individual components, and then carries out condensation at 40 to 90° C., per mole of phenol units present, with 0.5 to 4 mol of an aliphatic aldehyde with 1 to 6 C atoms, preferably formaldehyde, and—optionally—with 0.25 to 4 mol of urea.

A further preferred embodiment uses a crystallization modifier obtainable by reacting dihydroxydiphenyl sulfone with 0.5 to 5 mol of an aliphatic aldehyde having 1 to 6 C atoms, preferably formaldehyde, and 0.4 to 2 mol of sodium sulfite per mole of dihydroxydiphenyl sulfone at a temperature of 90 to 180° C.

Mixtures of the above-described condensation products can also be used.

The condensation products generally act as crystallization inhibitors.

The condensation product comprising sulfonate groups can be isolated from the resulting solutions by means, for example, of precipitation of the condensation product by addition of a water-miscible liquid which is not a solvent for the condensation product, or by evaporating the liquid reaction medium, by means of spray drying, for example.

In the finishing method of the invention the organic pigment is dissolved or dispersed in an aqueous solvent or mineral acid and crystallized from the solution or dispersion in the presence of the crystallization modifier. In one embodiment of the method of the invention the pigment is dissolved in concentrated sulfuric acid and crystallized by mixing the solution with an aqueous diluent. In that case the crystallization modifier may be present not only in the sulfuric acid solution of the pigment but also in the aqueous diluent. In a variant of this method the mixing is accomplished by combining the sulfuric acid pigment solution and the aqueous diluent using a mixing nozzle. The aqueous diluent here is generally water, and may comprise the crystallization modifier. Crystallization can also be accomplished by pouring the sulfuric acid pigment solution into water, preferably ice-water.

Generally speaking, the concentration of the pigment in the concentrated sulfuric acid is 1% to 30% by weight. The crystallization modifier is generally present in amounts of 0.1% to 30% by weight, based on the pigment. The volume of the aqueous diluent is generally from 1 to 12 times the volume of the sulfuric acid pigment solution.

In one variant of this embodiment the crystallization modifier is generated in situ in the sulfuric acid solution with the pigment. This is done by introducing the individual components of the condensation agent into the sulfuric acid pigment solution and carrying out condensation.

In a further variant of this embodiment the crystallized organic pigment, following crystallization, is aged in the presence of a surfactant. The surfactant may even be added to the aqueous diluent or may be added after the step of crystallization of the pigment suspension, and is generally in the form of an aqueous solution. The aging of the crystallized organic pigment takes place in general by stirring of the pigment suspension at temperatures of in general 40 to 100° C. over a period of 0.5 to 5 h. In the course of this time, larger pigment particles grow at the expense of smaller pigment particles and/or there is smoothing/healing of the crystal surface of the pigment particles. Since operation takes place in the presence of a surfactant, this procedure occurs as early as in the aqueous pigment suspension, containing sulfuric acid, with a comparatively low sulfuric acid content and a comparatively low temperature, of the kind obtaining after the mixing of sulfuric acid pigment solution with the aqueous diluent.

Suitable surfactants are the anionic, cationic, nonionic, and amphoteric surfactants specified below.

In another embodiment of the method of the invention the pigment is dispersed in dilute aqueous sulfuric acid and swollen in the presence of the crystallization modifier. Prior to the swelling operation it is advantageous to grind the pigment together with the crystallization modifier, in a ballmill with stirring mechanism, for example. It is particularly advantageous to grind the pigment, prior to swelling, together with the crystallization modifier and with a surface-active agent, more particularly a synthetic and/or natural resin such as rosin or hydrogenated, part-hydrogenated, disproportionated, dimerized or polymerized rosin. In the course of swelling, larger pigment particles grow at the expense of smaller pigment particles and/or there is a smoothing/healing of the crystal surfaces of the pigment particles. Suitable dilute sulfuric acid generally has a concentration of 50% to 85%, preferably 60% to 85% by weight. The swelling of the pigment in the dilute aqueous sulfuric acid in the presence of the crystallization modifier takes place in general at temperatures of from 15 to 90° C. over a time of in general 0.5 to 24 hours. The optimum swelling conditions can be ascertained for each type of pigment in preliminary experiments. Subsequently, dilution is carried out again with water. This is generally done by adding 2 to 6 times the amount of water to the pigment dispersion containing sulfuric acid. Subsequently, generally speaking, the dispersion is stirred for 0.5 to 2 hours more.

The swelling of the pigment can also be carried out as an aftertreatment step, preferably with pigment obtained by the above-described process of precipitation with an aqueous diluent, and isolated in solid form.

In a further embodiment of the method of the invention the pigment obtained by precipitation, for example, is dispersed in water and crystallized in the presence of the crystallization modifier and of a pigment solubility enhancer. As the solubility enhancer it is general practice to add an organic solvent. Suitable organic solvents are, for example, xylenes, glycols, alcohols, THF, acetone, NMP, DMF, and nitrobenzene. These solvents, based on the aqueous pigment suspension, are added in general in an amount of 0.1% to 50% by weight. The amount of crystallization modifier is generally 0.1% to 30% by weight, based on the aqueous pigment suspension (without organic solvent). In general the suspension is stirred in the presence of the organic solvent at temperatures in the range of from 15° C. to boiling temperature and thereafter the organic solvent is distilled off. Alternatively the method can also be carried out without solvent, at an elevated pressure. In that case, crystal growth of the pigment particles is directed by the crystallization modifier and occurs as a result of Ostwald ripening. The step can also be carried out as an aftertreatment step, using for example pigment which has been obtained by the above-described process of precipitation, with an aqueous diluent and isolated in solid form. In all cases the pigment crystallized in the presence of the crystallization modifier is subsequently isolated in solid form by filtration of the aqueous suspension.

In one variant of this embodiment a solution of the pigment in concentrated sulfuric acid is prepared and is mixed with water, preferably using a mixing nozzle. The suspension is stirred, generally at a temperature in the range from 15 to 90° C. over a period of 0.5 to 8 hours, and the pigment is isolated by filtration and washed preferably with water. The resulting pigment is suspended in water and crystallized in the presence of crystallization modifier and solubility enhancer. Suitable dispersants are, for example, pigment derivatives containing sulfonic acid groups, such as copper-phthalocyaninesulfonic acid.

Examples of suitable pigments which can be finished by the method of the invention include azo, azomethine, methine, anthraquinone, phthalocyanine, perinone, perylene, diketopyrrolopyrrole, thioindigo, thiazineindigo, dioxazine, iminoisoindoline, iminoisoindolinone, quinacridone, flavanthrone, indanthrone, anthrapyrimidine, and quinophthalone pigments. Preferred pigments are phthalocyanines, perylenes, quinacridones, indanthrones, quinophthalones, dioxazines, and diketopyrroles, more preferably phthalocyanines, perylenes, and indanthrones.

Preferred among the perylenes are the pigments of the C.I. Pigment Red 179 type. These pigments can be prepared by a variety of processes. For instance, C.I. Pigment Red 179 can be prepared by methylating perylene-3,4,9,10-tetracarboxylic diimide with a methylating agent or by condensing perylenetetracarboxylic anhydride with methylamine. The latter process is used with preference. An alternative is to use mixtures of the pigments prepared by these two methods. It is also possible to use pigment derivatives prepared by these methods, or mixtures of said derivatives.

During the crystallization, the crystallization modifier used in accordance with the invention is generally present in amounts of 0.1% to 30% by weight, preferably 0.3% to 25% by weight, more preferably 1% to 22% by weight, based on the pigment suspension. In certain cases it may be advantageous as well to use further crystallization modifiers, dispersants, surfactants or specialty polymers. Examples of further crystallization modifiers are imidazolemethyl- or pyrazolemethylquinacridone pigment sulfonic acids. Examples of suitable surfactants are anionic surfactants such as alkylbenzenesulfonates or alkylnaphthalenesulfonates or alkylsulfosuccinates, cationic surfactants such as quaternary ammonium salts, benzyltributylammonium chloride for example, or nonionic or amphoteric surfactants such as polyoxyethylene surfactants and alkyl or amidopropyl betaines.

Examples of suitable specialty polymers are homopolymers and copolymers of maleic acid, polyacrylic acid, polymethacrylic acid, polyurethanes, polyvinyl alcohol, polyvinylpyrrolidone or cellulose derivatives.

There may be further finishing steps afterward. Thus preferably the isolated pigment solid is blended with a pigment synergist. This is generally a derivative of an organic pigment or salt thereof, containing sulfonate groups or carbonate groups, or is a basic derivative of the aforementioned pigments. The pigment synergist is preferably a derivative of the pigment with which the synergist is blended. Generally speaking the pigment synergist is used in amounts of 0.1% to 15% by weight, preferably 0.5% to 10% by weight, based on the completed pigment formulation.

The average particle size of the finished pigments is generally in the range from 10 to 400 nm, preferably 20 to 200 nm.

The pigments finished by the method of the invention may comprise the crystallization modifier on the surface of the pigment particles. Also provided, therefore, are pigment preparations comprising particles of an organic pigment with a superficial covering of the pigment particles with the crystallization modifier used in accordance with the invention. Besides the pigment synergists already mentioned, the pigment preparations may include further additives, generally in amounts of up to 15% by weight. Examples of further additives are wetting agents, surfactants, antifoams, antioxidants, UV absorbers, stabilizers, plasticizers, and texturing assistants. These additives may be added as early as during the finishing step.

The pigment preparations are used for coloring and coating natural and synthetic materials.

The invention is elucidated in more detail by the examples which follow.

### EXAMPLES

The pigment preparations of the invention are tested in an aqueous varnish system.

For this purpose, first of all, an aqueous tinting paste is prepared on the basis of a water-dilutable polyurethane resin. 100 g of the polyurethane resin dispersion described in example 1.3 of WO-A-92/15405, 30 g of the pigment preparation and 50 g of water are suspended, adjusted to a pH of 8 using dimethylethanolamine, and ground in a ballmill (loaded with 1.0-1.6 mm SAZ beads [SAZ=silicon/aluminum/zirconium oxide]) for 4 h. In the second step 34 g of this aqueous tinting paste (15% by weight based on pigment) are added to 225 g of a polyurethane-based mixing varnish (described in example 3 of WO-A 92/15405). Following the addition of 7.5 g of water, a pH of 8 is set using aminoethanol. The suspension obtained is stirred at 1000 rpm with a propeller stirrer for 15 min.

Using the aqueous basecoat materials produced, metallic paints are prepared and applied by spraying.

### Example 1

Preparation of Crystallization Modifier K1

Crystallization modifier K1 is prepared according to example 2 of DE-A 101 40 551.

A pressure reactor is charged at room temperature with 1300 kg of deionized water together with 4100 kg (9.5 kmol) of technical-grade dihydroxydiphenyl sulfone (containing about 85% by weight of 4,4′-dihydroxydiphenyl sulfone, about 15% by weight of 2,4′-dihydroxydiphenyl sulfone, and small amounts of p-phenolsulfonic acid) in the form of a 60% strength by weight aqueous solution, and 1155 kg of a 30% strength by weight aqueous solution of formaldehyde (11.5 kmol) and 800 kg (3 kmol) of solid sodium sulfite (anhydrous) are added. A little 20% strength by weight aqueous sodium hydroxide solution is added to set a pH in the reaction mixture of 8 to 8.5. The pressure reactor is then closed and the reaction mixture is heated to 115° C. with stirring. After a short time at this temperature the reaction sets in. The temperature of the reaction mixture climbs to 150 to 160° C. and the pressure in the pressure reactor climbs to 4 to 5 bar (overpressure). External heating is used to maintain the temperature of the reaction mixture at 160° C. After the reaction mixture has been stirred at 160° C. for 3 hours, it is cooled to room temperature and admixed with 400 kg of sodium hydrogen sulfate. The solution obtained has a solids content of approximately 46% by weight.

### Example 2

140 parts of perylene-3,4,9,10-tetracarboxylic acid N,N′-dimethyldiimide (C.I. Pigment Red 179) are dissolved in 2576 parts of sulfuric acid (96% strength by weight) and the solution is conditioned to 25° C. The pigment preparation is subsequently precipitated from the acid with a solution at 25° C. of 56.4 parts of a 45.4% strength by weight solution of K1 in 10 000 parts of water, by nozzle precipitation using a Y-shaped nozzle (0.5 mm bores for the reactant flows), with the following precipitation parameters: water conveying rate 400 g/min, pigment solution conveying rate 100 g/min. The pigment is filtered, washed, and dried.

This gives a pigment preparation which, in an aqueous coating system based on a water-dilutable polyurethane resin, finishes a strongly colored, transparent paint finish with a yellowish red shade. The metallic finish is strongly colored and bright.

### Example 3

50 parts of the pigment preparation from example 2 are ground together with 2.5 parts of a perylene compound which contains sulfonic acid groups and has been prepared in accordance with EP 0 636 666 A2 (derivative (b2) from page 7 line 27 of EP 0 636 666 A2). The metallic finish comprising the pigment preparation is even more transparent and yellow than that of example 2.

### Example 4

50 parts of the pigment preparation from example 2 are ground together with 3.75 parts of a perylene compound containing sulfonic acid groups (prepared according to example 3 of EP 0 486 531 B1). The metallic finish of this pigment preparation is even more transparent and yellow than that of example 2.

### Example 5

140 parts of perylene-3,4,9,10-tetracarboxylic acid N,N′-dimethyldiimide (C.I. Pigment Red 179) and 61.7 parts of a 45.4% strength by weight solution of K1 are dissolved in 2576 parts of sulfuric acid (96% strength by weight) and the solution is conditioned to 25° C. The pigment preparation is subsequently precipitated from the sulfuric acid with water at 25° C., by nozzle precipitation using a Y-shaped mixing nozzle (0.5 mm bores for the reactant flows), with the following conveying rates: water 400 g/min, pigment solution in acid 100 g/min. This gives a precipitation suspension having a temperature of 60° C. The pigment preparation is filtered on a suction filter, washed to <100 μS (conductivity of the washing water), dried in vacuo in a drying oven at 80° C., and ground.

This gives a pigment preparation which, in an aqueous coating system based on a water-dilutable polyurethane resin, finishes a strongly colored, transparent paint finish with a yellowish red shade. The metallic finish is strongly colored and bright.

### Example 6

50 parts of the pigment preparation from example 5 are ground together with 3.75 parts of a perylene compound containing sulfonic acid groups (prepared according to example 3 of EP 0 486 531 B1). The metallic finish of this pigment preparation is even more transparent and yellow than that of example 5.

### Example 7

1000 parts of the suspension of pigment preparation 4 are admixed with 2.56 parts of a beta-naphthol ethoxylate (Lugalvan® BNO 12, BASF AG) and stirred at 60° C. for 5 hours. Thereafter the pigment preparation is filtered on a suction filter, washed to <100 μS, dried in vacuo in a drying oven at 80° C., and ground.

50 parts of this pigment preparation are ground together with 3.75 parts of a perylene compound containing sulfonic acid groups (and prepared according to example 3 of EP 0 486 531 B1). With high transparency and a yellowish red shade of the metallic finish, the aqueous coating system based on a water-dilutable polyurethane resin has a lower viscosity than that of example 6.

### Example 8

28.5 parts of perylene-3,4,9,10-tetracarboxylic acid N,N′-dimethyl diimide (C.I. Pigment Red 179) are dissolved together with 6.2 parts of a 45.4% strength by weight solution of K1 in 600 parts of 96% strength by weight sulfuric acid at 5-8° C. The solution is stirred at this temperature for 3 hours and then slowly introduced into 5 liters of ice-water. After 30 minutes of subsequent stirring the pigment is filtered off on a suction filter, washed to neutrality with water, dried in a forced-air oven, and ground.

This gives a pigment preparation which, in an aqueous varnish system based on a water-dilutable polyurethane resin, yields a strongly colored and transparent finish with a yellowish red shade. The metallic finish is strongly colored and brilliant.

### Example 9

100 parts of the pigment preparation from example 8 are ground together with 5 parts of a perylene compound containing sulfonic acid groups (and prepared according to example 3 of EP 0 486 531 B1). This gives a high-transparency preparation which in aqueous metallic varnishes exhibits a much more yellow-tinged shade than the pigment preparation of example 8.

### Example 10

50 parts of a perylene-3,4,9,10-tetracarboxylic acid N,N′-dimethyl diimide (C.I. Pigment Red 179) ground as per the prior art in a ball mill at 80° C. for 30 h are swollen together with 11.4 parts of a 44% strength by weight solution of K1 in 500 g of 77% strength by weight sulfuric acid at 50° C. for 16 hours. The batch is diluted with 2 l of ice-water, stirred for 30 minutes, and filtered, and the solid product is washed to neutrality and ground together with 3.75 parts of a perylene compound containing sulfonic acid groups (and prepared according to example 3 of EP 0 486 531 B1).

The preparation has a significantly higher transparency and a more yellow shade than a corresponding preparation prepared without the crystallization inhibitor.

### Example 11

95 parts of a perylene-3,4,9,10-tetracarboxylic acid N,N′-dimethyl diimide (C.I. Pigment Red 179) are ground together with 5 parts of dry K1 in accordance with the prior art in a ball mill at 50° C. for 50 h. 30 g of the dry-ground product are subsequently swollen in 300 g of 76% strength by weight sulfuric acid at room temperature for 16 hours. The batch is diluted with 2 l of ice-water, stirred for 30 minutes, and filtered, and the solid product is washed to neutrality and ground together with 3.75 parts of a perylene compound containing sulfonic acid groups (and prepared according to example 3 of EP 0 486 531 B1).

The preparation has a significantly higher transparency and a more yellow shade than a corresponding preparation prepared without the crystallization inhibitor.

### Example 12

80 g of perylene-3,4,9,10-tetracarboxylic acid N,N′-dimethyl diimide (C.I. Pigment Red 179, formula No. 71130), 10 g of resin (Dertopol®, DRT), and 4.2 g of dry K1 are ground on a vibratory mill at 50° C. for 50 h in a 0.75 l jacketed grinding container charged with 1.5 kg of steel balls (diameter 25 mm). 30 g of the dry-ground product are then swollen at room temperature for 16 hours in 300 g of 76% strength sulfuric acid. The batch is diluted with 2 l of ice-water, stirred for 30 minutes and filtered and the filter product is washed to neutrality and ground together with 3.75 parts of a perylene compound containing sulfonic acid groups (and prepared according to example 3 of EP 0 486 531 B1).

The pigment preparation from example 12 has a higher transparency and a more yellow and more chromatic shade than that from example 11. With outstanding rheological properties, moreover, it is easier to disperse in water-thinnable coating systems. On account of the high transparency, the pigment from example 12 is especially suitable for pigmenting metallic paints.

### Example 13

A solution of 80 g of indanthrone P.B. 60 in 987 g of 96% strength by weight sulfuric acid is stirred for two hours and during that time is conditioned at 25° C. This solution is run together via a mixing nozzle with a 0.3% strength by weight aqueous solution of K1, in a ratio of 2.5 parts of K1 solution to one part of pigment solution. During this procedure the temperature climbs to 60° C. The mixture is subsequently stirred at 60° C. for 30 minutes more. The suspension is filtered, washed with fully demineralized water to <100 μS, then dried in vacuo in a drying oven at 80° C. It is subsequently milled for 20 seconds in a coffee grinder at the highest speed setting. The resulting pigment has a high transparency and good flop—that is, a small goniochromatic color shift.

### Example 14

A pigment solution prepared as in example 12 is run together via a mixing nozzle with 2.5 parts of water. In the course of this procedure the temperature climbs to 60° C. The mixture is subsequently stirred at 60° C. for 30 minutes more. The suspension is filtered and the filtercake is washed with fully demineralized water to <100 μS. Subsequently the filtercake is again stirred up in fully demineralized water, and 10% by weight of K1, based on the pigment, is added. The suspension is introduced into a jacketed reactor where it is heated to 60° C. over the course of an hour. When at temperature, 20 g of p-xylene are added and the mixture is stirred for two hours. Xylene is distilled off then at 100° C.

The suspension is then filtered and the filtercake is washed with a little water and dried in a vacuum drying oven at 80° C. The pigment has high transparency and a high chroma.

### Example 15

With regard to the pigment solution prepared as in example 12, 4 g of Cu phthalocyanine are added. This solution is then run together via a mixing nozzle with 2.5 parts of water. In the course of this procedure the temperature climbs to 60° C. The mixture is subsequently stirred at 60° C. for 30 minutes more. The suspension is filtered and the filtercake is washed with fully demineralized water to <100 μS. Subsequently the filtercake is again stirred up in fully demineralized water, and 10% by weight of K1, based on the pigment, is added. The suspension is introduced into a jacketed reactor where it is heated to 60° C. over the course of an hour. When at temperature, 20 g of p-xylene are added and the mixture is stirred for two hours. Xylene is distilled off then at 100° C.

The suspension is then filtered and the filtercake is washed with a little water and dried in a vacuum drying oven at 80° C. The pigment has high transparency and a high chroma.

### Example 16

Preparation of Crystallization Modifier K2

Crystallization modifier K2 is prepared in accordance with example 1 of DE-A 101 40 551. 40 kmol of phenol heated to 60° C. are admixed over the course of 1 hour with 40 kmol of oleum having an SO₃ content of 24% by weight. The resulting mixture is heated to 160° C. and stirred at that temperature for 4 hours. Then it is allowed to cool to 80° C., 25 kmol of urea in the form of a 68% strength by weight solution are incorporated with stirring, and then 40 kmol of formaldehyde in the form of a 30% strength by weight aqueous solution are metered in over the course of 1.5 hours. The mixture obtained is subsequently admixed with about 34 kmol of NaOH in the form of a 50% strength by weight aqueous solution, giving a pH of 4.5.

The dihydroxydiphenyl sulfone content is 10% by weight, based on the dry weight.

### Example 17

140 parts of perylene-3,4,9,10-tetracarboxylic acid N,N′-dimethyldiimide (C.I. Pigment Red 179) and 40 parts of K2 (as a 35% strength by weight solution) are dissolved in 2576 parts of sulfuric acid (96% strength by weight) and the solution is conditioned to 25° C. The pigment preparation is subsequently precipitated from the sulfuric acid solution with water at 25° C., by nozzle precipitation using a Y-shaped mixing nozzle (0.5 mm bores for the reactant flows), with the following conveying rates: water 400 g/min, pigment solution in acid 100 g/min. This gives a precipitation suspension having a temperature of 60° C. The pigment preparation is filtered on a suction filter, washed with water, dried in vacuo in a drying oven at 80° C., and subsequently ground.

### Example 18

50 parts of the pigment preparation from example 16 are ground with 3.75 parts of the perylene compound containing sulfonic acid groups and prepared according to example 3 of EP 0 486 531 B1. This gives a pigment preparation which, in an aqueous coating system based on a water-dilutable polyurethane resin, finishes a strongly colored, transparent paint finish with a yellowish red shade. The metallic finish is strongly colored and bright.

1. A method of finishing an organic pigment that comprises dissolving or dispersing the pigment in an aqueous solvent or a mineral acid and crystallizing the pigment from the solution or dispersion in the presence of a crystallization modifier, and subsequently isolating the pigment as a solid, wherein the crystallization modifier is a condensation product containing sulfonate groups and formed from at least one hydroxyarylsulfonic acid and/or at least one hydroxydiaryl sulfone compound and at least one aliphatic aldehyde having 1-6 C atoms, optionally urea, and optionally an alkali metal sulfite, or is a mixture of such condensation products.

2. The method according to claim 1, wherein the crystallization modifier is a condensation product of at least one hydroxyarylsulfonic acid, at least one aliphatic aldehyde having 1-6 C atoms, and optionally urea.

3. The method according to claim 1, wherein the crystallization modifier is a condensation product of at least one hydroxydiaryl sulfone compound, at least one aliphatic aldehyde having 1-6 C atoms, and an alkali metal sulfite.

4. The method according to claim 1, wherein the hydroxyarylsulfonic acid is phenolsulfonic acid and the hydroxydiaryl sulfone compound is dihydroxydiphenyl sulfone.

5. The method according to any claim 1, wherein the aliphatic aldehyde is formaldehyde.

6. The method according to claim 1, wherein the pigment is dissolved in concentrated sulfuric acid and crystallized by mixing the solution with an aqueous diluent, and the crystallization modifier is contained in the sulfuric acid solution or in the aqueous diluent.

7. The method according to claim 6, wherein the crystallized organic pigment, before being isolated as a solid, is aged in the presence of a surfactant.

8. The method according to claim 7, wherein the surfactant is present in the aqueous diluent or is added after the crystallization step.

9. The method according to claim 1, wherein the pigment is dispersed in dilute aqueous sulfuric acid and swollen in the presence of the crystallization modifier.

10. The method according to claim 1, wherein the pigment is dispersed in water and crystallized in the presence of the crystallization modifier and of a pigment solubility enhancer.

11. The method according to claim 10, wherein the pigment solubility enhancer is selected from xylenes, glycols, alcohols, THF, acetone, NMP, DMF, and nitrobenzene.

12. The method according to claim 1, wherein the isolated pigment solid is blended with a pigment synergist which is a derivative comprising sulfonate or carbonate groups or is a basic derivative of an organic pigment.

13. The method according to claim 12, wherein the pigment synergist is a derivative of the pigment.

14. The method according to claim 1, wherein the pigment is selected from the group consisting of azo, azomethine, methine, anthraquinone, phthalocyanine, perinone, perylene, diketopyrrolopyrrole, thioindigo, thiazine indigo, dioxazine, iminoisoindoline, iminoisoindolinone, quinacridone, flavan throne, indanthrone, anthrapyrimidine, and quinophthalone pigments.

15. A pigment preparation comprising particles of organic pigment with a superficial covering of the pigment particles with the crystallization modifier as defined in claim 1.

16-17. (canceled)
